(19) United States
(12) Patent Application Publication
(10) Pub. No.: US 2012/0289711 A1
(43) Pub. Date: Nov. 15, 2012

(54) METHOD FOR PRODUCING CARBON NANOMATERIALS AND/OR CARBON MICROMATERIALS AND CORRESPONDING MATERIAL

(75) Inventors: Jens Helbig, Nuernberg (DE); Christian Zenkel, Gesees (DE)

(73) Assignee: FUTURECARBON GMBH, Bayreuth (DE)

(21) Appl. No.: 13/505,252

(22) PCT Filed: Oct. 25, 2010
(86) PCT No.: PCT/EP2010/066062

§ 371 Date: Jul. 16, 2012

(30) Foreign Application Priority Data
Oct. 28, 2009 (DE) 10 2009 051 084.2

(51) Int. Cl. C01B 31/02; C01B 7/01; C01B 21/00; C07D 207/448; C07D 307/89; C07C 237/48; C07C 63/66; C07C 233/65; C07C 63/72; B01J 19/12; C07C 63/42; B82Y 99/00; B82Y 40/00

(52) U.S. Cl. 548/548; 204/157.6; 204/157.94; 204/157.82; 549/234; 562/488; 564/155; 562/405; 564/180; 562/853; 562/840; 977/847; 977/752

(57) ABSTRACT

The present invention relates to a method for producing carbon nanomaterials and/or carbon micromaterials, in particular multi-wall carbon nanotubes. This method is characterized according to the invention in that the materials, in particular the side walls of the materials, undergo microwave-assisted functionalization. In addition, a correspondingly modified material is described.

The present invention first relates to a method for producing carbon nanomaterials and/or carbon micromaterials, in particular multi-wall carbon nanotubes. In addition, the invention relates to a carbon nanomaterial and/or carbon micromaterial, in particular multi-wall carbon nanotubes.

In particular, the present invention relates to a covalent side-wall functionalization of single-wall or multi-wall carbon nanotubes (CNTs).

For better wetting of CNTs and a possible covalent crosslinking of CNT surfaces to a reactive matrix surrounding them in order to improve mechanical properties, it is necessary to chemically modify the surfaces of CNTs.

Materials improved thereby, which are based on crosslinking and thermoplastic plastics, are possible for a wide range of applications, such as, for example, fiber composite components with improved interlaminar shearing strength, elastomers with elevated E-modulus, highly crosslinked resins with increased toughness, mechanically reinforced polyamides and the like.

In addition, improved dispersions based on aqueous or organic solvents can also be produced with surface-modified CNTs, and these dispersions then can be utilized as precursors for coatings, as additives in polymers, metals or ceramics.

In principle, CNT surfaces can be modified in different ways, such as:
Application of so-called surfactants, primarily tensides, which are bound to the surface of CNTs via Van der Waals interactions;
By coordinated polyaromatic compounds via a π-π interaction on the surface;
By growth of polymers on the surface of CNTs via “grafting from” methods;
By depositing metal or metal-oxide particles or films onto the CNT surface;
By oxidation of the CNT surface with oxidizing acids and further functionalization of the carboxyl groups that form;
By microwave-assisted cyclo-additions with enophilic and dienophilic reactants, the CNTs themselves reacting as the dienophiles or the enophiles.
By introducing molecules of low molecular weight with a specific terminal group via reactions that lead to a covalent binding to the CNT surface.

The two last-named methods are of particular interest for the application of CNTs as reinforcing material in polymers, since here the strongest binding between the polymer matrix and the CNT-filler particles can be assured. Only by such a strong binding is it possible to obtain the reinforcing mechanisms known from composite material teaching, for example, force transfer to the embedded particles. In addition, the particular structure of CNTs is not too strongly attacked, as is the case with an oxidation of the surface, for which reason, both the mechanical properties as well as the electronic properties can be retained for the most part.

The next-to-last of the named methods (microwave-assisted cyclo-additions) is above all suitable for a smooth side-wall functionalization of CNTs without damaging the σ-system, with the targeting of a carboxyl group. The thus-functionalized CNTs can be further derivatized and functionalized without problem via known methods and are suitable for a plurality of applications, for example, in resins. Thus, for example, covalent crosslinking of CNTs with polymer matrices, and therefore, an improvement in the mechanical and electronic properties of resins and of other polymers can be achieved.

Another advantage of this method lies in the fact that, due to the selection of suitable reactants, the π-system and thus the electronic properties remain largely undisturbed, which again makes these thus-functionalized CNTs interesting for electronic applications.

The modification of CNTs with covalently bound molecules has been investigated for several years in different ways and has also already been utilized commercially for specific functionalities.

First, a distinction must be made between the functionalization of single-wall carbon nanotubes (SWCNTs) and multi-wall carbon nanotubes (MWCNTs). This is because there are fundamental differences in the type of reaction mechanisms that can be utilized at the present time for SWCNTs or MWCNTs, respectively.

A direct side-wall functionalization has currently only been demonstrated definitively for SWCNTs. This is explained by the stronger reactivity of SWCNTs based on the greater curvature of their surfaces and thus also by a greater pyramidalization of the π-system.

Oxidizing by means of the previous methods, such as with HNO₃, for example, without seriously disrupting the properties of CNTs is only possible for MWCNTs. The oxidizing of SWCNTs by means of oxidizing acids or gases without too greatly damaging or completely destroying SWCNTs is currently not possible.

Non-covalent functionalizations previously could be definitively demonstrated only for SWCNTs.

At the present time, most studies on covalent functionalization of CNTs in the literature describe SWCNTs. The reasons for this are not generally known, but it is assumed that the great curvature of their surfaces and the strong pyramidalization of the π-system of SWCNTs resulting therefrom makes them particularly reactive, and thus first makes possible a chemical reaction of the π-bonds of the aromatic rings.

The reactions described for SWCNTs have previously not been definitively demonstrated for a direct side-wall functionalization of MWCNTs.

The current publications have up to now demonstrated a functionalization only for a reaction in which the CNT reacts as a dienophile. This has been previously proven analytically also only for SWCNTs and not for MWCNTs.

It was shown in more recent studies that the radius of curvature has an influence on the reaction, and the reactivity and thus the possibility of a covalent side-wall functionalization based on the radius of curvature would be very different for SWCNTs and MWCNTs . The radius of curvature for SWCNTs is clearly greater than for MWCNTs. Therefore, the previously described reactions, which had been demonstrated for SWCNTs, cannot be applied to MWCNTs under the given maladaptive conditions without further research, due to their clearly lower reactivity, which is attributable to the smaller radius of curvature of the external end wall of the tube.

Microwave reactions per se have still not been previously applied in industry to CNTs and have also been known in the literature only for reactions in which the CNTs themselves react as dienophiles.

For this reason, a multi-step pathway to functionalization has been previously usually carried out for MWCNTs. First, the surface of the MWCNTs is oxidized, for the most part, by means of concentrated nitric acid. In this process, of course, the π- and σ-bond system of the graphene layers is attacked and sensitively disrupted thereby, which has as a consequence a clear worsening of the mechanical properties and the chemical stability of the MWCNTs as well as a damaging of the same. This damaging also acts on layers found deeper inside, since this type of oxidizing, after disrupting the first side wall, also extends beyond it to the wall lying thereunder and also damages it. Thus, an uncontrolled disruption of the CNT on very defective regions of the CNT until the tube breaks is possible at this site.

At these oxidized sites, which are essentially composed of carboxyl groups, reactive centers are then created via another multi-step process by substitution of the carboxyl groups with a halogen compound, for example, thionyl chloride, and more functional terminal groups can be introduced on these reactive centers.

The type of direct side-wall functionalization that is possible for SWCNTs is not possible with the same efficacy for MWCNTs, due to the lower reactivity of the MWCNT surface. Also, the routes used for MWCNTs can often be carried out with strongly etching reagents, for example, thionyl chloride, and only with high cost for apparatus, for example, protective gas and necessary additional occupational safety measures due to the toxicity of the reagents.

Reaction routes that can directly functionalize the side wall without completely disrupting the sidewall, and can manage without highly toxic reagents and expensive apparatus are currently under investigation exclusively for SWCNTs.

Proceeding from the named prior art, the problem of the present invention is to provide a method for producing carbon nanomaterials and/or carbon micromaterials, in particular multi-wall carbon nanotubes, as well as a carbon nanomaterial and/or carbon micromaterial, in particular, multi-wall carbon nanotubes, in which the above-named disadvantages can be avoided.

The problem is solved according to the invention by the method with the features according to the independent patent claim 1, as well as the material with the features according to the independent patent claim 8. Further features and details of the invention can be taken from the subclaims, the description and the drawings. Features and details that are described in connection with the method thus also apply, of course, in connection with the material and vice versa, so that relative to the disclosure of one aspect of the invention, reference is always made each time to the full extent to the disclosure of the other aspect of the invention.

By means of the present invention, in particular, a microwave-assisted, especially covalent, side-wall functionalization of multi-wall carbon nanotubes (MWCNTs) can be carried out, for example, with maleic anhydride, and advantageously by a Diels-Alder reaction, in order to produce functional acid groups. In particular, the present invention relates to a covalent side-wall functionalization of single-wall or multi-wall carbon nanotubes (CNTs).

According to the present invention, in particular, microwave-assisted methods are described for producing molecules that are introduced covalently on the side wall of an MWCNT and that can be provided with selected functional terminal groups, without disrupting the side wall.

“Functional group” particularly involves a group that determines the material properties and/or the reaction behavior of compounds that bear it.

These methods particularly make use of the reaction-accelerating properties of microwave radiation, which in particular makes it possible to shorten to just a few minutes the long reaction times of 24-48 hours known from the classical reaction for Diels-Alder reactions.

Likewise, this method represents a reaction of MWCNTs with a dienophilic reactant.

According to the first aspect of the invention, a method is provided for producing carbon nanomaterials and/or carbon micromaterials, in particular multi-wall carbon nanotubes, which is characterized in that the materials, in particular the side walls of the materials, undergo microwave-assisted functionalization.

Carbon nanomaterials and carbon micromaterials, in particular, are microscopically small structures based on carbon, for example, composed of carbon. The size of carbon nanomaterials thus particularly lies in the nanometer range, while the size of carbon micromaterials particularly lies in the micrometer range.

The materials can be introduced to functionalization under microwave irradiation.

The materials can preferably be provided in a reaction mixture, the reaction mixture being introduced to functionalization under microwave irradiation, at least temporarily. The reaction mixture advantageously may be composed of two or more components, one of the components being a carbon nanomaterial and/or a carbon micromaterial.

A covalent, microwave-assisted side-wall functionalization of MWCNTs with enophiles such as maleic anhydride, maleimide and their derivatives, for producing functional, terminal-position primary amino, hydroxyl and acid groups, as well as functional groups that leave the conjugated π-system nearly intact, can be advantageously achieved on the surface of the CNT side wall by the pathway that will be described in more detail in the following.

The functionalization can be achieved advantageously by an effect of microwave irradiation on the reaction mixture, an effect known in theory that shortens the reaction time of reactions in organic molecular chemistry, particularly for cyclo-additions.

For the microwave functionalization, preferably MWCNTs or SWCNTs, which are purified in particular, are provided dry or as a dispersion with maleic anhydride or one of its derivatives, such as those named further below in Table 1, for example. Such a dispersion particularly involves an above-described reaction mixture.

For example, water or different higher and high-boiling organic solvents, such as 1,5-pentanediol, 1,4-butanediol, ethanol, butanol, toluene, DMF, THF and others, can be used as the solvent for the dispersions.

The concentration of CNTs in the solvent can advantageously amount to between 0.001 and 10 weight percent, the invention not being limited to these values. Only a sufficiently good segregating of the CNTs in the dispersion with a simultaneous lower viscosity of the dispersion, for example 10 mPa.s to 1 Pa.s, is important for facilitating a stirring of the dispersion.

The dispersion is preferably mixed with the maleic anhydride or its derivative by means of classical dispersion and mixing methods, in order to obtain a mixing that is as homogeneous as possible. The ratio of maleic acid derivative to CNTs can, of course, be selected as desired in this case, an excess of the maleic acid derivative being required for the reaction.

After this, the reaction mixture can preferably be flushed with protective gas, for example with CO₂, N₂, Ar, He, Ne, or the like.

The material or the reaction mixture can preferably be rotated under microwave irradiation with vigorous stirring and/or by a non-static suspension of the reaction vessel in the microwaves.

Subsequently the reaction mixture is brought to reaction, preferably under microwave irradiation, preferably within 1 to 20 minutes, preferably in a temperature range of 50-300° C. and preferably kept at this temperature for 2 to 120 minutes, the method advantageously being constantly controlled by means of temperature sensors inside and outside the reaction vessel. Preferably, the material or the reaction mixture can be brought to reaction for a defined time period, preferably for 1 to 20 minutes under microwave irradiation. In another embodiment, the material or the reaction mixture can be brought to reaction in a defined temperature range, preferably in a temperature range of 50 to 300° C.

In this case, the reaction mixture is preferably continuously stirred and/or moved in a rotating reaction vessel and advantageously flushed with protective gas.

The described microwave method is preferably conducted with the above-named protective gases, in order to prevent plasma formation and an excess reacting and removal of the maleic anhydride or its derivative with air oxygen. However, the method may also be conducted successfully without first removing the oxygen.

Subsequently, the reaction mixture is advantageously cooled to a temperature between 10° C. and 30° C. The reaction product is advantageously washed with a large amount of pure solvent, such as, for example, H₂O, methanol, ethanol, butanol, toluene, DMF, THF, or the like and can be subsequently dried.

After this purification step, the product can be boiled advantageously once more in H₂O, for example for 0.5 to 10 hours, in order to open the maleic anhydride and to obtain the dicarboxylic acid, whereby this process also represents a smooth alternative for the σ-system and thus the structure of CNTs, for producing carboxylic acid-functionalized CNTs.

The material or the reaction mixture can preferably be functionalized by means of a Diels-Alder reaction. The Diels-Alder reaction is a chemical reaction in which bonds are built up between carbon atoms.

An example of embodiment is described below for the microwave functionalization.

MWCNTs, which are purified in particular, are mixed dry with dry, finely sieved maleic anhydride in a reaction flask for a microwave-assisted cyclo-addition. Subsequently, the powder mixture, which may involve a reaction mixture, in microwaves, is provided with a stirrer and flushed with He.

The mixture is subsequently heated to 200° C. with constant stirring within 20 min. and then kept at this temperature for approximately 10 min.

After cooling the reaction mixture to room temperature, the powder is washed with a large amount of ethanol and subsequently dried.

In order to obtain the dicarboxylic acid functionalities, the purified product must then be boiled in H₂O for up to 2 h and subsequently dried.

The described method for microwave functionalization of multi-wall and single-wall carbon nanotubes (MWCNTs and SWCNTs) represents a working covalent side-wall functionalization by means of microwave irradiation, in which CNTs are present as enophiles and react with a dienophile.

Table 1 shows different hydrides that are considered as reaction partners that can be used for the reaction.

TABLE 1

| | |
|---|---|
| Maleimide | Maleic anhydride |
| Maleic acid | N-(4-hydroxyphenyl)-maleimide |
| Maleic acid dichloride | Maleic acid diamide |
| Propenoic acid | Propenoic acid amide |
| Propenoic acid chloride | 2-Butene diacid |
| 2-Butene diacid chloride | 2-Butene diacid amide |

The reactions were successfully conducted in batches of 50 milligrams up to 10 grams and the reaction times amounted to 2 to 120 minutes. In contrast to previous methods for carboxylation with HNO₃, the forming product offers the advantage of a COOH functionalization of carbon nanotubes with minimal damage to the CNT structure, whereby the mechanical properties of the functionalized MWCNTs are for the most part maintained. The reaction also makes possible the addition of maleimide and other maleic acid derivatives and thus, for example, producing terminal-position amino groups or initiator molecules for polymerizations on the surface of CNT side walls, by means of which a broad range of other applications, above all in the field of composites, is opened up.

The reaction mechanism itself takes place according to the Diels-Alder cyclo-addition known in the literature and was previously described in the literature by Delgado et al. only for a dienohilic reaction on the part of MWCNTs and not for an enophile as in this case.

In contrast to the previously familiar methods for producing carboxyl groups on the side walls of carbon nanotubes, a Diels-Alder cyclo-addition only opens up the π-system of CNTs and is not reliant on an opening of and thus a consequent disruption of the σ-bond framework.

This method additionally does not require the presence of defects in the framework structure of the carbon nanotubes used, as is the case for the oxidation of the CNT side wall with oxidizing acids such as HNO₃.

The method according to the invention preferably has one or more of the features named in the Claims, the Description, the Examples, the Tables and the Drawings.

According to a second aspect of the invention a carbon nanomaterial and/or carbon micromaterial, in particular multi-wall carbon nanotubes, is provided, which is characterized in that the material, in particular the side walls of the material, is/are functionalized with microwave support.

Advantageously, the material can be produced, has been produced, or is producible with a method according to the invention, as described above.

Advantageously, the material has one or more of the features named in the Claims, the Description, the Examples, the Tables and the Drawings.

The invention will be explained in more detail based on an embodiment example with reference to the attached drawing. Here:

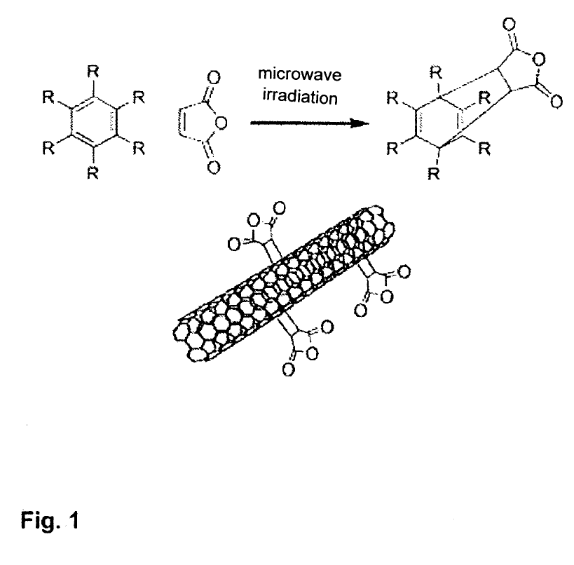

FIG. 1 shows the reaction pathway of the microwave-assisted covalent side-wall functionalization.

The product that forms is shown schematically in FIG. 1, in order to further clarify the type of bond and the different possibilities.

1. A method for producing carbon nanomaterials and/or carbon micromaterials, in particular multi-wall carbon nanotubes, is hereby characterized in that the materials, in particular the side walls of the materials, undergo microwave-assisted functionalization.

2. The method according to claim 1, further characterized in that the materials for the functionalization are placed under microwave irradiation.

3. The method according to claim 1, further characterized in that the materials are provided in a reaction mixture and that for the functionalization the reaction mixture is placed under microwave irradiation, at least temporarily.

4. The method according to claim 1, further characterized in that the material or the reaction mixture can be brought to reaction under microwave irradiation for a defined time period, preferably for 1 to 20 minutes.

5. The method according to claim 1, further characterized in that the material or the reaction mixture is brought to reaction in a defined temperature range, in particular a temperature range from 50 to 300° C.

6. The method according to claim 1, further characterized in that the method is conducted with protective gas.

7. The method according to one of claims 1 to 6, further characterized in that the material or the reaction mixture is functionalized by means of a Diels-Alder reaction.

8. A carbon nanomaterial and/or carbon micromaterial, in particular multi-wall carbon nanotubes, is hereby characterized in that the material, in particular the side walls of the material, undergo microwave-assisted functionalization.

9. The material according to claim 8, further characterized in that it is produced, has been produced or can be produced with a method characterized in that the materials, in particular the side walls of the materials, undergo microwave-assisted functionalization.
